(19) United States
(12) Patent Application Publication
(10) Pub. No.: US 2015/0202432 A1
(43) Pub. Date: Jul. 23, 2015

(54) ELECTRODE STRUCTURE FOR DEEP BRAIN STIMULATION

(71) Applicant: PACESETTER, INC., Sylmar, CA (US)

(72) Inventors: Zoltan Somogyi, Simi Valley, CA (US); Gene A. Bornzin, Simi Valley, CA (US)

(73) Assignee: PACESETTER, INC., Sylmar, CA (US)

(21) Appl. No.: 14/161,544

(22) Filed: Jan. 22, 2014

(51) Int. Cl. A61N 1/05; H01B 13/06

(52) U.S. Cl. CPC A61N 1/0534; H01B 13/06

(57) ABSTRACT

An electrode structure is incorporated into an implantable lead for deep brain stimulation. The electrode structure comprises multiple radial electrode elements. The radial electrode elements of the electrode structure are separated by holes (e.g., slots) and held together by end rings of the electrode structure. The electrode elements are separated from one another at a later stage of a manufacturing process through the use of centerless grinding that removes the end rings.

### TECHNICAL FIELD

This application relates generally to implantable devices and more specifically, but not exclusively, to an electrode structure used for deep brain stimulation.

### BACKGROUND

Deep brain stimulation (DBS) is used to treat numerous disorders such as Parkinson's disease, tremors, dystonia, depression, obsessive compulsive disorder, and epilepsy. Of these disorders, Parkinson's disease is the most commonly treated malady. For example, Ventral Intermediate Nucleus (VMI) stimulation, Subthalamic Nucleus (STN) stimulation, and Globus Pallidus internal (GPi) stimulation have been shown to be effective in treating Parkinson's disease. DBS reduces Levodopa-induced dyskinesias, including involuntary movements and painful cramping.

DBS is applied to a patient through the use of an implantable lead. A hole, called a burr hole, is drilled in the patient's skull. A thin test lead (e.g., less than 1 mm in diameter) including mapping electrodes is inserted through the hole and directed to a target area of deep brain tissue. The position of the distal end of the lead is manipulated, as necessary, to identify the exact location within the brain where stimulation is to be applied. This process involves, for example, determining the location of the mapping electrodes lead (e.g., using computed tomography (CT) scanning or magnetic resonance imaging (MRI)) and performing trial stimulations using the electrodes to determine whether the stimulation has the desired effect. After confirming the desired stimulation location within the brain tissue, the test lead is removed and a DBS stimulation lead (e.g., having a diameter of 1.5 mm or less) including stimulation electrodes is implanted in the patient so that the stimulation electrodes are positioned at the desired stimulation location. The implanted DBS stimulation lead is connected to an implanted stimulation device that generates stimulation signals that are coupled to the electrodes via lead conductors. Thus, under the control of the stimulation device, prescribed stimulation therapy is provided to the patient via the electrodes to treat the brain disorder.

In general, it is desirable to use leads of a very small diameter for deep brain stimulation. For example, a typical lead has a diameter of less than 1.5 mm. However, such a small diameter tends to make construction of a lead difficult. For example, it may be relatively difficult to manufacture a lead with multiple or complex electrodes since the electrodes are very small.

### SUMMARY

A summary of several sample aspects of the disclosure follows. This summary is provided for the convenience of the reader to provide a basic understanding of such aspects and does not wholly define the breadth of the disclosure. This summary is not an extensive overview of all contemplated aspects, and is intended to neither identify key or critical elements of all aspects nor delineate the scope of any or all aspects. Its sole purpose is to present some concepts of one or more aspects in a simplified form as a prelude to the more detailed description that is presented later. For convenience, the term some aspects may be used herein to refer to a single aspect or multiple aspects of the disclosure. Similarly, the term some embodiments may be used herein to refer to a single embodiment or multiple embodiments.

The disclosure relates in some aspects to an electrode structure used for deep brain stimulation. The electrode structure is incorporated into an implantable lead whereby, upon implant, stimulation signals applied to the electrode structure causes stimulation energy to radiate to adjacent brain tissue to treat a brain disorder.

The electrode structure comprises multiple electrode elements positioned around a longitudinal axis. Thus, upon implant, the direction at which stimulation energy radiates from the electrode structure can be controlled by selectively applying stimulation signals to one or more of the electrode elements.

In general, the electrode structure is very small. For example, the electrode structure may have a diameter of 1.5 mm or less. To make the electrode elements and implantable lead easier to manufacture, the electrode structure is initially formed as one piece. After the electrode structure is incorporated into the implantable lead, the electrode structure is machined (e.g., by centerless grinding) to separate the individual electrode elements. A dedicated stimulation signal may thus be applied to each of the electrode elements, thereby providing the desired directionality for the application of stimulation energy to brain tissue.

The disclosure relates in some aspects to a method of manufacturing an implantable lead. In some aspects, the method of manufacture involves incorporating a unitary electrode structure, comprising radially-oriented electrode elements (e.g., segments), into a cylindrical lead. The radial electrode elements of the electrode structure are separated by holes (e.g., slots) and held together by end rings of the electrode structure. Separate electrical conductors (e.g., wires) are connected to each electrode element. The electrode elements are then separated from one another at a later stage of the manufacturing process. For example, once the electrode structure is embedded in lead body material, centerless grinding may be used to remove the end rings.

In view of the above, an electrode structure and/or an implantable lead constructed in accordance with the teachings herein may take various forms. Several non-limiting examples follow.

In some aspects, an electrode structure constructed in accordance with the teachings herein comprises: a hollow cylindrical body comprising an electrically conductive metal, wherein: the body defines a plurality of holes, such that a middle section and end sections of the body are delimited, at least in part, by the holes; each of the end sections has an interior diameter that is greater than an interior diameter of the middle section; the middle section comprises a plurality of electrode elements separated by the holes; and each electrode element comprises a top surface and two side edges.

In some aspects, an implantable lead constructed in accordance with the teachings herein comprises: a lead body comprising insulation material; and a plurality of electrode elements embedded at least partially within the insulation material, wherein: each electrode element comprises a top surface, two side edges, and two arcuate end edges, each side edge of each electrode element comprises a recessed ledge that has a top surface that is below an adjacent portion of the top surface of the electrode element, and, for each of the recessed ledges, at least a portion of the insulation material lies between the top surface of the recessed ledge and an outer surface of the lead body.

In some aspects, a method of manufacturing an implantable lead in accordance with the teachings herein comprises: removing a portion of an interior surface of each of the end sections of a cylindrical electrode structure, such that each of the end sections has an interior diameter that is greater than an interior diameter of the middle section of the cylindrical electrode structure; forming a plurality of holes in the middle section, such that the middle section comprises a plurality of electrode elements separated by the holes, wherein each electrode element comprises a top surface and two side edges; for each of the electrode elements, connecting an electrical conductor to the electrode element; forming a lead body by embedding the electrode structure, at least in part, in an insulation material; and centerless grinding the embedded electrode structure, such that the end sections are removed from the embedded electrode structure.

### BRIEF DESCRIPTION OF THE DRAWINGS

These and other aspects of the disclosure will be more fully understood when considered with respect to the following detailed description, the appended claims, and the accompanying drawings, wherein:

FIG. 1 is a simplified oblique view of an embodiment of an electrode structure;

FIG. 2 is a simplified end view of the electrode structure of FIG. 1;

FIG. 3 is a simplified end view of the electrode structure of FIG. 1 after centerless grinding of the electrode structure;

FIG. 4 is a simplified oblique view of the electrode structure of FIG. 1 after centerless grinding of the electrode structure;

FIG. 5 is a simplified plan view of a spindle upon which the electrode structure of FIG. 1 may be mounted;

FIG. 6 is a simplified end view of the spindle of FIG. 5;

FIG. 7 is a simplified oblique view of an embodiment of a spindle assembly that includes the spindle of FIG. 5 and electrode structures of FIG. 1;

FIG. 8 is a simplified end view of the spindle assembly of FIG. 7;

FIG. 9 is a simplified oblique view of the spindle assembly of FIG. 7 after polymer reflow;

FIG. 10 is a simplified plan view of the spindle assembly of FIG. 9 after centerless grinding;

FIG. 11 is a simplified oblique view illustrating a polymer reflow process on the spindle assembly of FIG. 7;

FIG. 12 is a simplified plan view of an embodiment of a molding process employing an electrode structure as taught herein;

FIG. 13 is a simplified diagram of an embodiment of an electrode structure comprising three electrode elements;

FIG. 14 is a simplified diagram of another embodiment of an electrode structure comprising three electrode elements;

FIG. 15 is a simplified diagram of another embodiment of an electrode structure comprising three electrode elements;

FIG. 16 is a simplified flowchart of an embodiment of operations that may be performed to manufacture an implantable lead comprising an electrode structure as taught herein; and

FIG. 17 is a simplified diagram of an embodiment of a medical system comprising a stimulation device connected to an implantable lead to provide DBS for a patient.

In accordance with common practice, the various features illustrated in the drawings may not be drawn to scale. Accordingly, the dimensions of the various features may be arbitrarily expanded or reduced for clarity. In addition, some of the drawings may be simplified for clarity. Thus, the drawings may not depict all of the components of a given apparatus or method. Finally, like reference numerals may be used to denote like features throughout the specification and figures.

### DETAILED DESCRIPTION

The description that follows sets forth one or more illustrative embodiments. It will be apparent that the teachings herein may be embodied in a wide variety of forms, some of which may appear to be quite different from those of the disclosed embodiments. Consequently, the specific structural and functional details disclosed herein are merely representative and do not limit the scope of the disclosure. For example, based on the teachings herein one skilled in the art should appreciate that the various structural and functional details disclosed herein may be incorporated in an embodiment independently of any other structural or functional details. Thus, an apparatus may be implemented or a method practiced using any number of the structural or functional details set forth in any disclosed embodiment(s). Also, an apparatus may be implemented or a method practiced using other structural or functional details in addition to or other than the structural or functional details set forth in any disclosed embodiment(s).

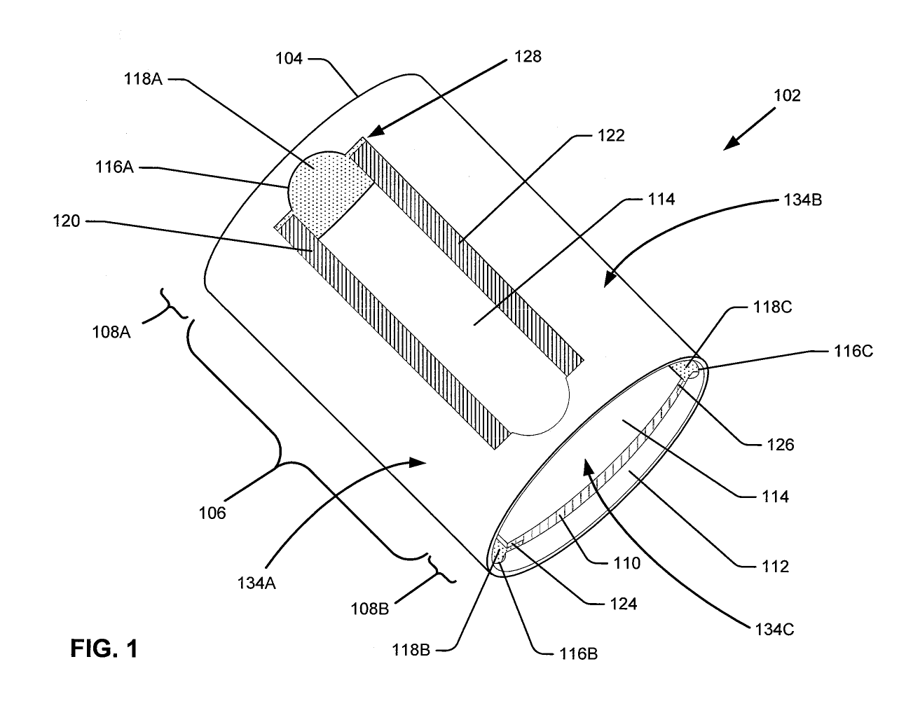

FIG. 1 is a simplified example of an embodiment of an electrode structure 102. The electrode structure 102 is constructed of an electrical conductor material. In addition, the electrode structure 102 is typically a biocompatible material to facilitate implant within a patient (e.g., in cases where the electrode is not coated with a biocompatible material). For example, the electrode structure 102 may comprise platinum (e.g., a platinum-iridium material), or some other suitable material.

In the example of FIG. 1, the electrode structure 102 comprises a hollow cylindrical body 104. The body 104 includes a middle section 106 that takes the form of a smaller cylinder and two end sections 108A and 108B that each take the form of a ring. An explicit delineation between the middle section 106 and the end sections 108A and 108B is not shown in FIG. 1 since the body 104 is a unitary body in this example. It should be appreciated, however, that the body 104 need not be a unitary body.

The interior diameter of each of the end sections 108A and 108B is greater than the interior diameter of the middle section 106. This difference in interior diameter can be seen for the end section 108B which shows that there is a step up 110 (the substantially vertical surface with the hatched lines) from a substantially horizontal interior surface 112 of the end section 108B to a substantially horizontal interior surface 114 of the middle section 106.

The middle section 106 includes three holes that separate the middle section 106 into three electrode elements once the end sections 108A and 108B are removed, as discussed in detail below. Slot 116A delineated at its ends by two half circles is an example of such a hole. Such a slot may be formed, for example, by machining (e.g., with a round bit) the middle section 106 along a longitudinal path. FIG. 1 also illustrates the ends of two other slots 116B and 116C that are formed in the middle section 106, but that are mostly hidden from view in FIG. 1

In some embodiments, the slots 116B and 116C take the same form as the slot 116A and all three slots are equidistant from one another. In this case, if the body 104 was rotated along its longitudinal axis, so that the slot 116B or the slot 116C was in the position of the slot 116A in FIG. 1, FIG. 1 would look that same in that case. It should be appreciated, however, that the slots need not be identical and/or equidistance in all embodiments.

Also, it should be appreciated that a different number of slots (or more generally, holes) may be used in different implementations. Thus, different embodiments may utilize a different number of individually controllable electrode elements.

For purposes of illustration, dot patterns 118A, 118B, and 118C are used to indicate that if a person looked through the corresponding slot from the view of FIG. 1, the person would see whatever background happens to be behind the body 104. To better understand the view of FIG. 1, the dot pattern 118A may be contrasted with the section of the interior surface 114 of the middle section 106 that is visible through the slot 116A.

A recessed ledge is formed along each longitudinal side of each slot 116A, 116B, and 116C. For the slot 116A, the top surfaces of ledges 120 and 122 are represented by hatched lines. FIG. 1 also depicts one ledge 124 for the slot 116B and one ledge 126 for the slot 116C.

As shown in FIG. 1, there are gaps under the ends of the ledges 124 and 126 and by the gaps (e.g., gap 128) above the ends of the ledges 120 and 122. These gaps indicate that the top surface of each ledge lies below the interior surfaces of the end sections 108A and 108B. Here, the “top” surface of a ledge refers to the surface of the ledge towards the outer surface of the body 104, while the top surface being “below” an interior surface means that the top surface is closer to the hypothetical central longitudinal axis of the body 104 (axis not shown) than the interior surface.

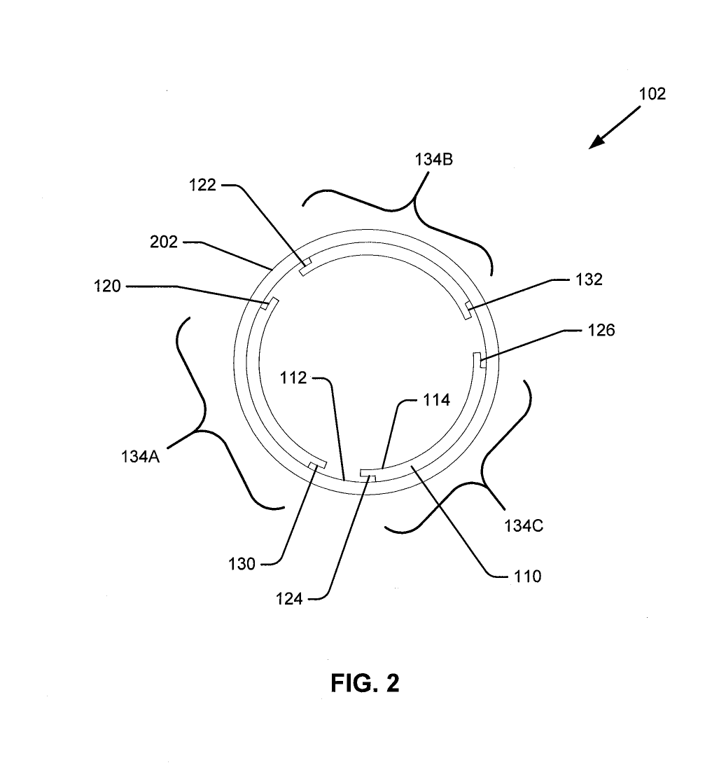

All of the ledges in the electrode structure 102 are illustrated in FIG. 2 which depicts a view from the end of the body 104 that includes the end section 108B. Specifically, FIG. 2 shows side views of the ledges 120, 122, 124, and 126 of FIG. 1, as well as ledges 130 and 132 that are not visible from the view of FIG. 1. For reference, this figure also depicts an outer surface 202 of the body 104, the interior surface 112 of the end section 108B, the interior surface 114 of the center section 106, and the step 110 between the interior surfaces 112 and 114.

In view of the above, it can be seen that FIGS. 1 and 2 show a unitary electrode structure 102 having three radial electrode elements 134A, 134B, and 134C. These electrode elements also may be referred to as electrode segments. The radial electrode elements 134A, 134B, and 134C are separated (e.g., delineated) by slots 116A, 116B, and 116C. Again, the slots 116B and 116C are only partially visible in the oblique view of FIG. 1.

From the view of FIG. 2, it can be seen that each of the radial electrode elements 134A, 134B, and 134C includes two recessed ledges, one ledge extending from each longitudinal side edge. Also, each of the radial electrode elements 134A, 134B, and 134C includes two arcuate end edges, only one of which is visible from the view of FIG. 2 for each electrode element. As discussed in more detail below, the ledges serve to hold each electrode element in place when the electrode assembly 102 is embedded in a lead body material such as a reflowed polymer (e.g., Optim).

When the electrode structure 102 is in the form of FIGS. 1 and 2, the end sections 108A and 108B hold the three radial electrode elements 134A, 134B, and 134C together. The radial electrode elements 134A, 134B, and 134C can thus be separated by removing the end sections. In accordance with the teachings herein, this may be achieved by removing a portion of the outer surface of the body 104. For example, grinding the outside of the body 104 using a centerless grinder to a specified depth would remove a part of the outer surface of the radial electrode elements 134A, 134B, and 134C, while removing the end sections 108A and 108B entirely.

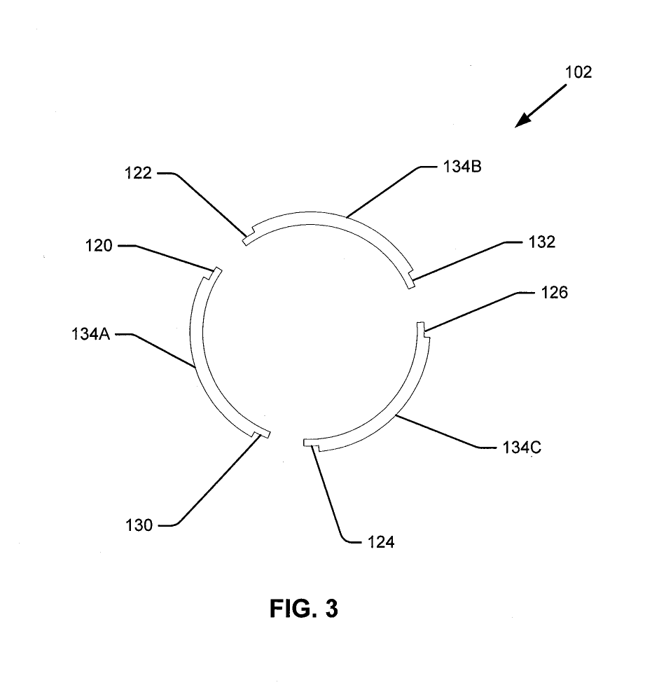

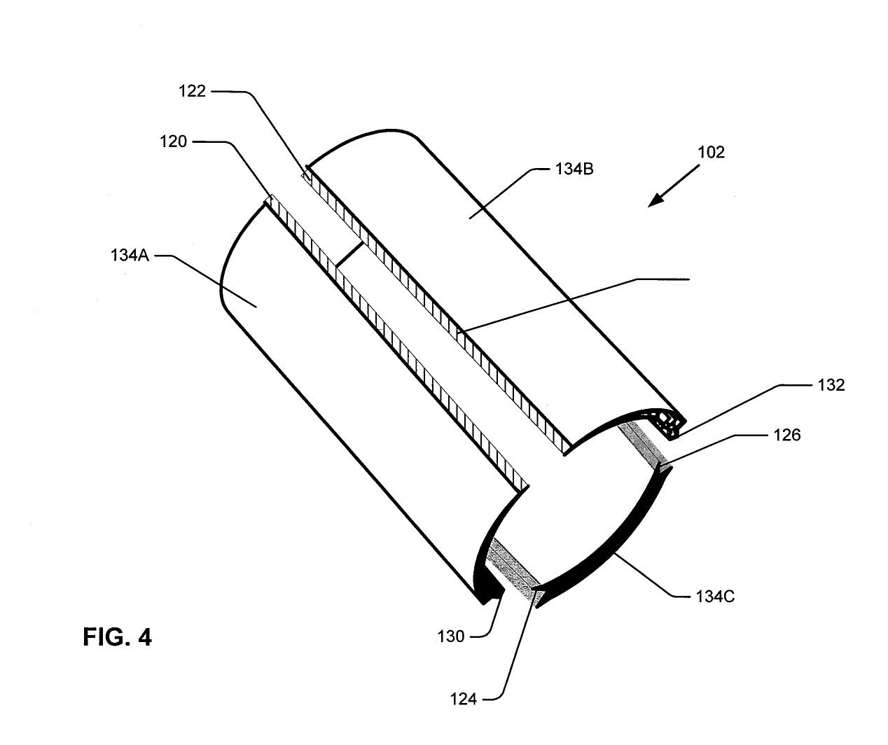

A simplified example of the result of such a centerless grinding operation is illustrated in FIGS. 3 and 4. FIG. 3 is taken from the same end view as FIG. 2. FIG. 4 is taken from the same oblique view as FIG. 1. In FIGS. 3 and 4, the three radial electrode elements 134A, 134B, and 134C from the center section 106 (FIG. 1) remain, but they are not longer attached to one another. Thus, the electrode elements 134A, 134B, and 134C are now electrically independent.

For purposes of explanation, the electrode elements 134A, 134B, and 134C are shown as being suspended in the same orientation that the electrode elements 134A, 134B, and 134C were in before the end sections 108A and 108B were removed. As discussed in more detail below, when the electrode elements 134A, 134B, and 134C are incorporated into an implantable lead, the electrode elements will be embedded in, and thus held in place by, a material of the implantable lead body.

In some embodiments, the electrode structure 102 is incorporated into a lead through the use of a support structure upon which the electrode structure 102 is placed prior embedding the electrode structure 102 in a material of the lead (e.g., prior to reflowing the polymer). That is, a support structure may be used as a core for mounting the electrode structure 102. For convenience, such a support structure may be referred to as a spindle in the discussions that follow.

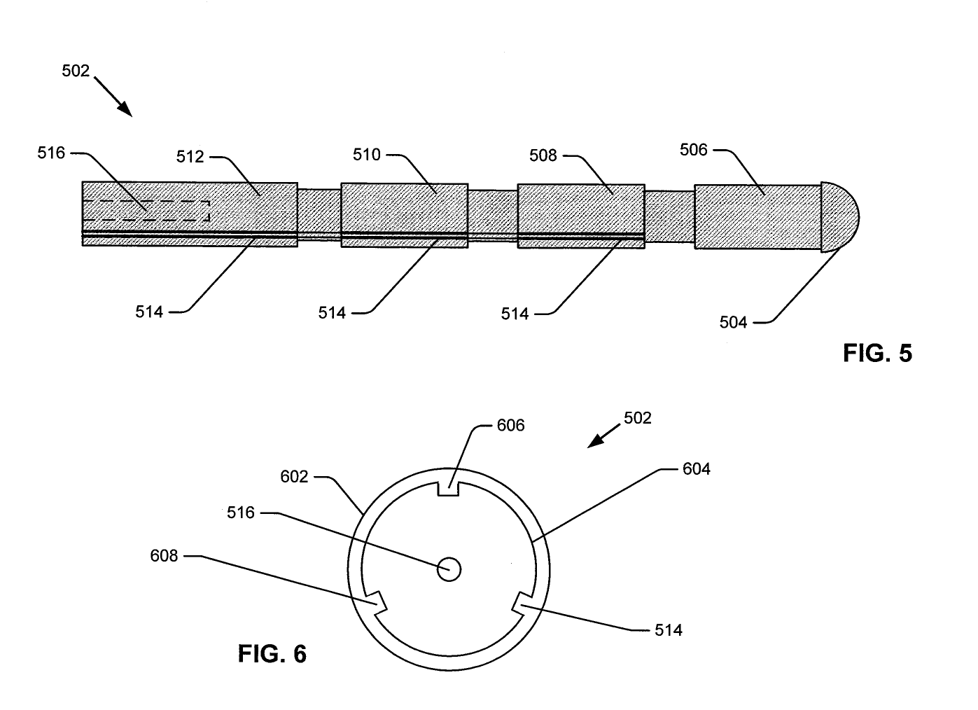

FIG. 5 illustrates an embodiment of a spindle 502. The spindle 502 may be constructed of, for example, polyether ether ketone (PEEK), polyurethane, or some other suitable material.

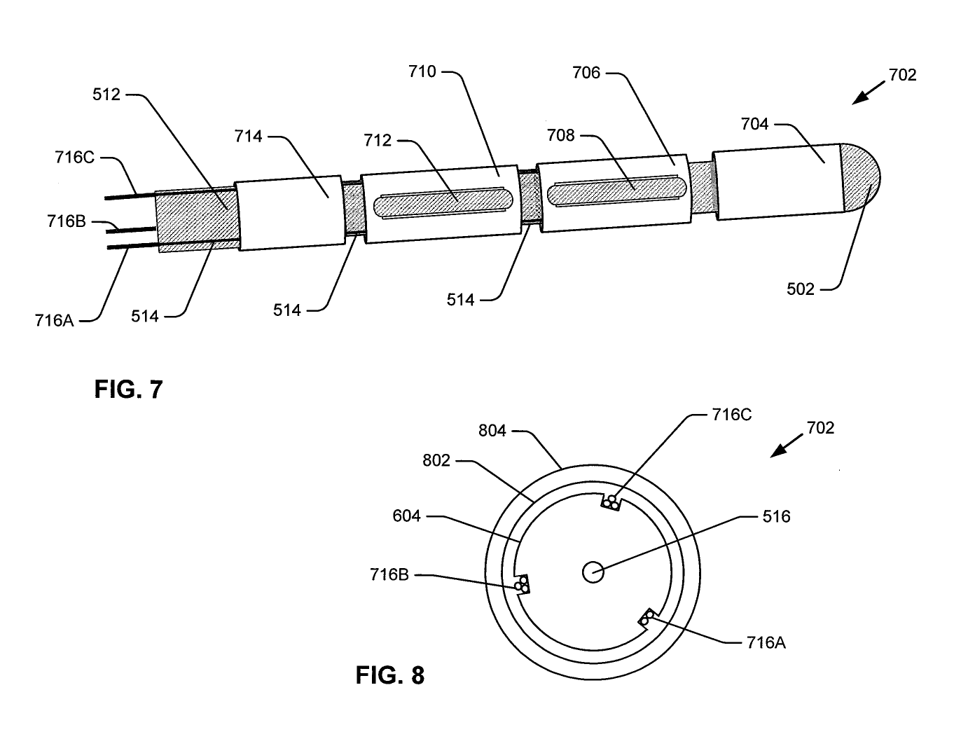

As indicated in FIG. 7 below, the spindle 502 is constructed to enable hollow cylindrical electrodes to be slid onto the spindle 502. From the view of FIG. 5, the electrodes would be slid onto the left side of the spindle 502 and the moved to the right. The spindle 502 includes an end section 504 to prevent an electrode from sliding off the spindle 502 to the right. The spindle 502 also includes specific sections 506, 508, 510, and 512 upon which different electrodes will rest.

Furthermore, the spindle 502 includes several channels (e.g., grooves) for routing electrical conductors to the electrodes. In the view of FIG. 5, only one channel 514 is shown.

Finally, the spindle 502 includes a stylet hole 516 as represented by the dashed lines in FIG. 5. For example, a hole may be drilled down the longitudinal axis of the spindle 502. This hole may then be used for placing a stylet (not shown) during implant of an implantable lead that includes the spindle 502.

FIG. 6 illustrates an end view of the spindle 512 as seen looking at the spindle 512 from the left side of the view of FIG. 5. Here, it can be seen that the outer diameter 602 of the end section 504 is wider than the outer diameter 604 of the other sections (e.g., sections 506, 508, 510, and 512) of the spindle 502. FIG. 6 also illustrates an end view of the channel 514 as well as two other channels 606 and 608 that are not seen in the view of FIG. 5. Finally, FIG. 6 also shows the stylet hole 516 as seen from the end view.

FIG. 7 illustrates a spindle assembly 702 where electrodes have been positioned on the spindle 502. A unitary electrode 704 (e.g., a simple ring electrode) has been placed on the section 506 (not visible in FIG. 7). An electrode structure 706 in accordance with the teachings herein (e.g., the electrode structure 102) has been placed on the section 508 (partially visible through the slot 708 in the electrode structure 706). An electrode structure 710 in accordance with the teachings herein has been placed on the section 510 (partially visible through the slot 712 in the electrode structure 710). A unitary electrode 714 (e.g., a simple ring electrode) has been placed on the section 512 (partially visible in FIG. 7). The electrodes may be adhesively attached to the spindle 502 using epoxy or some other suitable adhesive.

FIG. 7 also illustrates several electrical conductors that are connected to the electrodes. For example, dedicated electrical conductors may be connected to respective ones of the three electrode elements that partially comprise a given electrode structure 706 or 710. Also, dedicated electrical conductors may be connected to respective ones of the two unitary electrodes 704 and 714. Thus, in an embodiment employing eight electrical conductors, one electrical conductor will connect to the electrode 704, three electrical conductors will connect to the electrode structure 706, three electrical conductors will connect to the electrode structure 710, and one electrical conductor will connect to the electrode 714. To reduce the complexity of FIG. 7, only a few representative conductors 716A, 716B, and 716C are shown.

An electrical conductor may be connected to an electrode in various ways. In some embodiments, a given electrical conductor is welded (e.g., laser welded) to the inside of the electrode (or electrode element).

FIG. 7 also illustrates that the electrical conductors may run along the channels in the spindle 502. For example, electrical conductors 716A are indicated as running within the channel 514.

FIG. 8 further illustrates the channels of the spindle 502 and the routing of electrical conductors through these channels. FIG. 8 is an end view of the spindle assembly 702 as seen looking at the spindle assembly 702 from the left side of the view of FIG. 7. Here, it can be seen that the electrical conductors 716A are routed through the channel 514, the electrical conductors 716B are routed through the channel 608 (FIG. 6), and the electrical conductors 716C are routed through the channel 606 (FIG. 6).

FIG. 8 also illustrates that the outer diameter 802 of the electrode 714 is wider than the outer diameter 604 of the spindle section 512. In addition, FIG. 8 illustrates that in some embodiments, the outer diameter 804 of the electrode structure 710 (and/or electrode structure 706) is wider than the outer diameter 802 of the electrode 714. This latter relationship may exist, for example, in cases where the electrode structures 706 and 710 are to be subjected to centerless grinding, but the electrodes 704 and 714 are not (e.g., to reduce material waste).

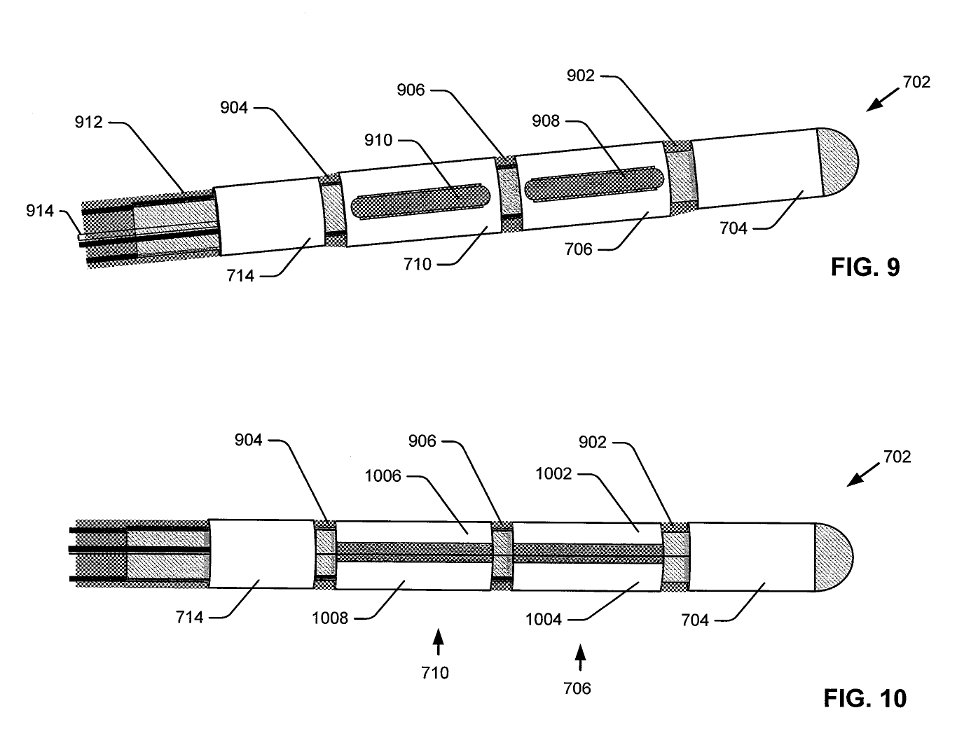

Referring now to FIG. 9, in conjunction with incorporating the spindle structure 702 into an implantable lead, the spindle structure 702 is subjected to a reflow process or some other suitable process whereby the electrode structures are embedded within a suitable lead body material. This material serves to hold the electrode elements in place and to electrically insulate the electrode elements from one another. Typically, the material is a biocompatible material (e.g., in cases where the implantable lead is not subsequently coated with a biocompatible material). Examples of such a material include Optim, Pellethane polyurethane, or Bionate. For convenience, this material may be referred to as an insulation material in the discussion that follows.

Several locations of the insulation material are labeled in FIG. 9. Insulation material 902 has filled in the space between the electrode 704 and the electrode structure 706. As indicated by the non-flat transition of the insulation material 902 between these electrodes, the electrode structure 706 may have a larger outside diameter than the electrode 704 prior to centerless grinding. A similar transition is shown for the insulation material 904 that has filled in the space between the electrode 714 and the electrode structure 710. Insulation material 906 has filled in the space between the electrode structures 706 and 710. It should be appreciated that the outer portion of insulation material 902, 904, and 906 will be removed as part of the centerless grinding process along during the removal of the outer portion of the electrode structures 706 and 710.

FIG. 9 further illustrates that insulation material has filled in the slots (holes) of the electrode structures. As specifically illustrated, insulation material 908 has filled in one of the slots of the electrode assembly 706 and insulation material 910 has filled in one of the slots of the electrode assembly 710. It should be appreciated that other insulation material will have filled in the other slots of the electrode structure (not shown in FIG. 9).

Of note, some of this insulation material will be above the top of the recessed ledges of the individual electrode elements. Thus, the insulation material will serve to hold the electrode elements in place on the implantable lead after centerless grinding has removed the end sections of each electrode structure.

FIG. 9 also illustrates that insulation material 912 may fill in proximal (left side, in the view of FIG. 9) sections of the spindle structure 702. In various embodiments, this insulation material may be attached to the lead body at some point during the manufacturing process (e.g., after centerless grinding). Alternatively, the lead may be formed during a reflow process or some other suitable process, such that the lead and spindle assembly are constructed as a unitary structure.

In some embodiments, a tube 914 is employed to maintain or form the stylet lumen during the reflow process. Such a tube may be constructed of, for example, Bionate, Polytetrafluoroethylene (PTFE), or Ethylene tetrafluoroethylene (ETFE).

To reduce the complexity of FIG. 9, the electrical conductors are shown as being routed in a linear manner to the left of the spindle assembly 702. In practice, however, the electrical conductors would typically be wrapped in a spiral or a coil (e.g., around the stylet lumen tube 914). Coiling the electrical conductors in this manner provides the lead body with homogeneous bending properties when the lead is bent along its axis. This may prevent, for example, axial displacement of the lead tip in the brain when the lead is locked into the burr hole plug fixed in the patient's skull.

FIG. 10 depicts the spindle structure 702 of FIG. 9 after centerless grinding. As shown, centerless grinding reduces the lead's overall radius, removes the outer shell of the electrode assemblies 706 and 710, and separates each unitary electrode assembly into separate radial electrode elements.

The unitary electrode assembly 706 of FIG. 9 has thus been separated into three radial electrode elements after the centerless grinding process. Similarly, after the centerless grinding process, the unitary electrode structure 710 of FIG. 9 has been separated into three radial electrode elements. In the view of FIG. 10, only the electrode elements 1002 and 1004 are depicted for the electrode structure 706, and only the electrode elements 1006 and 1008 are depicted for the electrode structure 710. The remaining electrode segments are not visible from the view of FIG. 10.

Some insulation material and some of the material of other electrodes also may be removed during the centerless grinding process. As indicated in FIG. 10, some of the insulation material on the outer periphery of the insulation material 902, 904, and 906 has been removed (e.g., the transitions between electrodes are now uniformly flat). In some embodiments, the ring electrodes 704 and 714 also may be radially downsized due to the centerless grinding operation. For example, the ring electrodes 704 and 714 may be slightly oversized to ensure that the finished lead, after centerless grinding, will have a uniform outer circumference.

In view of the above, it should be appreciated that a unitary electrode structure as taught herein can be handled as a single unit while manufacturing an implantable lead. This electrode structure can then be separated into two or more electrode elements by centerless grinding during a later stage of the manufacturing process. The process of centerless grinding removes the outer surface from the unitary electrode structure to separate the electrode elements after the electrode elements have been embedded in a lead body material (e.g., an insulator material).

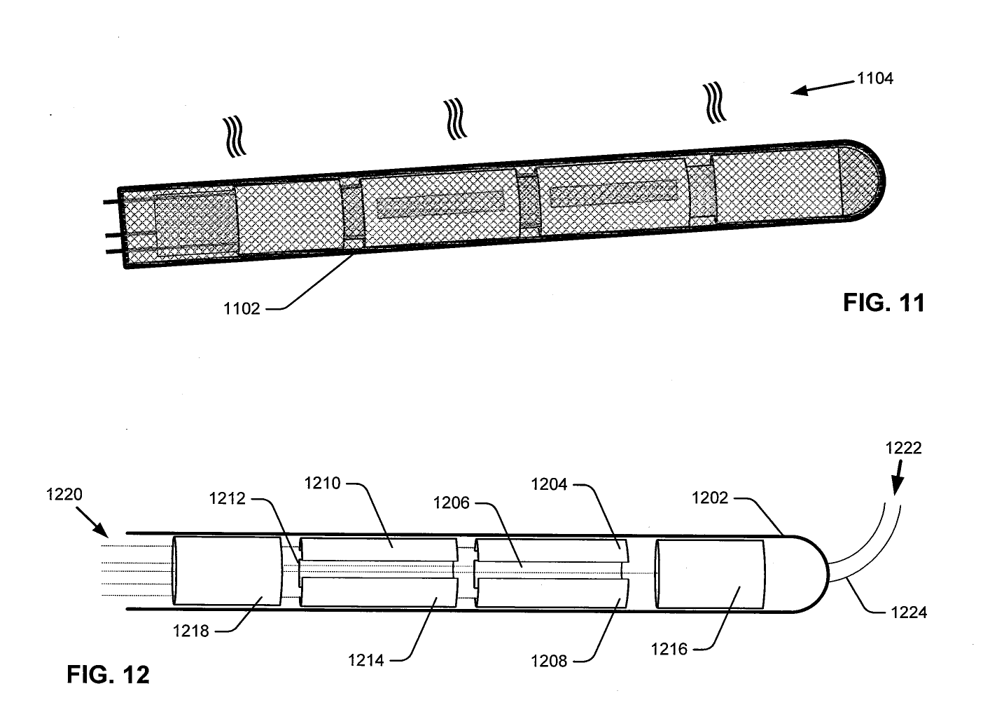

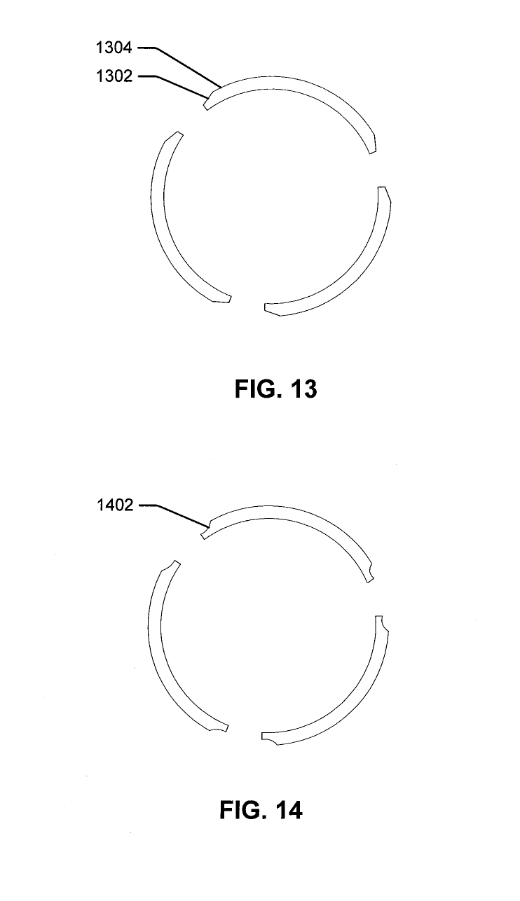

Different techniques may be used to embed the electrode elements in the lead body material. Two representative examples are described in FIGS. 11 and 12.

FIG. 11 illustrates an example of a process similar to the process described above where a polymer-like polyurethane or other suitable material is reflowed to fill in the spaces between the electrodes.

The body of a lead may be formed by inserting a stylet lumen tube filled with a mandrill (not shown) into the lumen hole described above, and then slipping a polyurethane tube over the stylet lumen tube and the electrical conductors. Optionally, the electrical conductors could be wrapped around a stylet lumen tube.

Next, shrink tubing 1102 is placed over the polyurethane as shown in FIG. 11. The shrink tube is heated (as indicated by heat lines 1104) to shrink the tubing and reflow the polyurethane around the wires, the stylet lumen tube, and all of the electrodes. Finally, the structure is centerless ground to separate the unitary structure into the electrode segments and to make the lead isodiametric.

FIG. 12 illustrates an example of a process where a mold 1202 is used to make the distal tip of a lead including the electrodes. As shown in FIG. 12, electrode elements 1204, 1206, and 1208 corresponding to a first electrode structure, electrode elements 1210, 1212, and 1214 corresponding to a second electrode structure, and ring electrodes 1216 and 1218, along with attached electrical conductors 1220 are placed in the mold 1202. For example, the mold 1202 may open to accept these components.

In one example of a molding process, the electrodes and electrical conductors may then be insert molded in place by injecting a mold material 1222 such as a thermoplastic (e.g., PEEK), polyurethane, or some other suitable material into the mold via an injection port 1224. The lead body can then be fused to the distal tip by reflowing if the lead body is made of a similar material or the same material (e.g., polyurethane) that was used for the insert molding.

In another example of a molding process, the electrodes and electrical conductors may be inserted into a silicone rubber mold. The mold can then be filled with an epoxy such as Hysol.

An electrode segment as taught herein may take different forms in different implementations. For example, a different shape may be employed for the recessed ledge in some implementations, while other implementations may omit the recessed ledge (e.g., other techniques may be employed to affix an electrode element to a lead body).

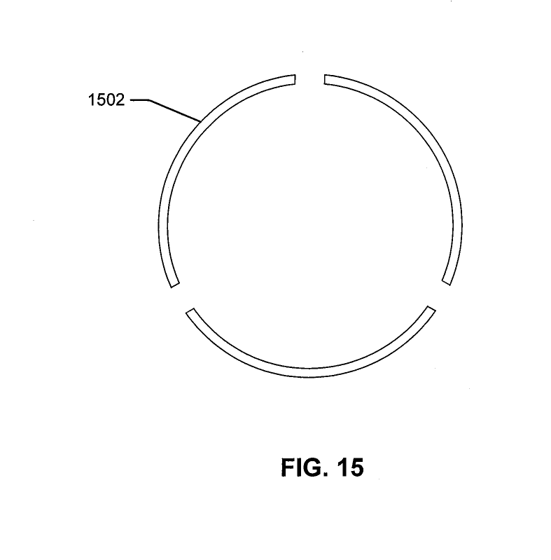

FIGS. 13 and 14 illustrate different shapes that may be employed for the recessed ledge. In FIG. 13, the top surface 1302 of the recessed ledge forms an angle with the adjacent top surface 1304 of the electrode structure. In FIG. 14, the top surface 1402 of the recessed ledge has a slight radius. FIG. 15 illustrates an embodiment where the segments 1502 of the electrode structure do not have recessed ledges.

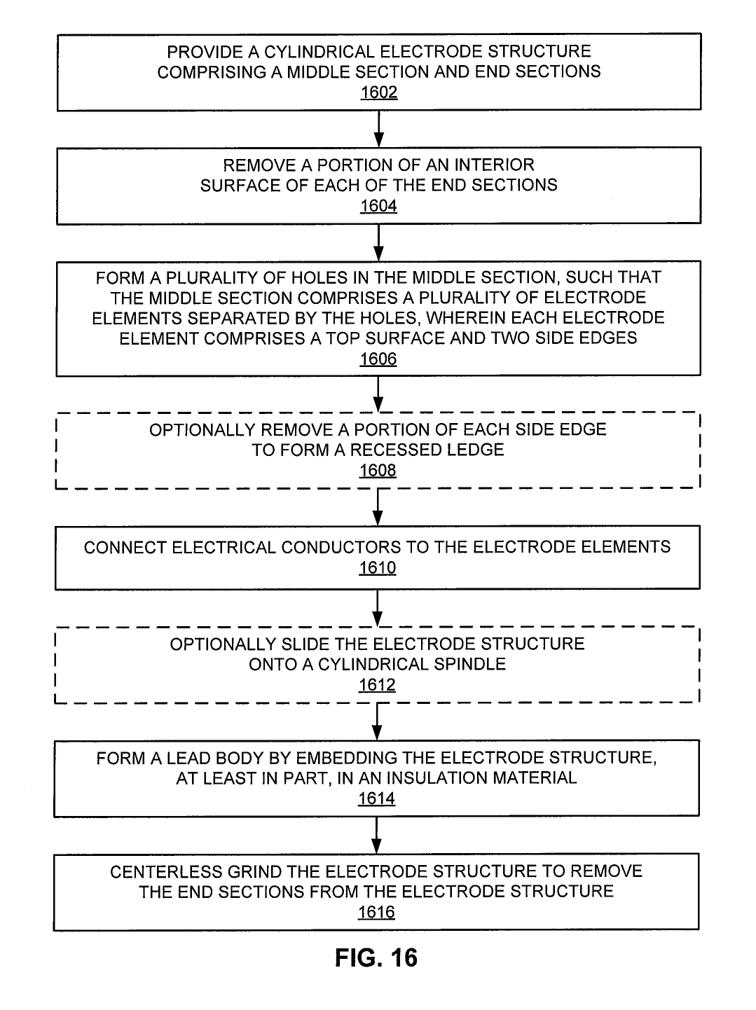

FIG. 16 illustrates an example of a method for manufacturing an implantable lead that incorporates an electrode structure as taught herein. For convenience, the operations of FIG. 16 (or any other operations discussed or taught herein) may be described as being performed by specific components. It should be appreciated, however, that these operations may be performed by other types of components and may be performed using a different number of components. It also should be appreciated that one or more of the operations described herein may not be employed in a given implementation.

Blocks 1602-1608 relate to making a unitary electrode structure. For example, a machining process may be employed where the electrode structure is constructed in four steps. Briefly, this process may involve obtaining a hollow cylinder, machining slots and narrow indentations into the cylinder, and boring out the insides of the ends of the cylinder out to create the end rings that hold the radial electrode elements together. Advantageously, the electrode structure can be machined using standard methods.

As represented by block 1602, a cylindrical electrode structure is provided (e.g., manufactured or purchased). As discussed herein, the electrode structure comprises a middle section and end sections.

As represented by block 1604, a portion of an interior surface of each of the end sections is removed. A sufficient amount of material is removed so that each of the end sections has an interior diameter that is greater than an interior diameter of the middle section as discussed herein.

As represented by block 1606, several holes are formed in the middle section. As a result, the middle section will comprise a plurality of electrode elements separated by the holes, wherein each electrode element comprises a top surface and two side edges. Thus, in some aspects, these holes serve to delimit the middle and end sections of the body.

As represented by optional block 1608, for each of the side edges of each of the electrode elements, a portion of the side edge may be removed. In this way, a recessed ledge is formed having a top surface that is below an adjacent portion of the top surface of the electrode element.

Blocks 1610-1616 relate to making an implantable lead. As discussed above, this process may involve a reflow process, a mold process, or some other suitable process.

As represented by block 1610, for each of the electrode elements, an electrical conductor is connected to the electrode element. For example, one end of an electrical conductor may be laser welded to an inner surface of an electrode segment.

As represented by optional block 1612, in implementations that employ a spindle, the electrode structure is slid onto the spindle. In addition, if the spindle defines at least one channel running longitudinally along an outer portion of the spindle, the electrical conductors may be routed through the at least one channel.

As represented by block 1614, a lead body is formed by embedding the electrode structure, at least in part, in an insulation material. In some embodiments, forming the lead body involves reflowing insulation material over the electrode structure. In some embodiments, forming the lead body involves placing the electrode structure into a mold, and injecting the insulation material into the mold. As discussed herein, for each of the recessed ledges of the electrode structure, at least a portion of the insulation material lies between the top surface of the recessed ledge and an outer surface of the lead body. In this way, each of the electrode elements of the electrode structure may be securely embedded in the insulation material.

As represented by block 1616, a centerless grinding process is used on the embedded electrode structure. Consequently, the end sections are removed from the embedded electrode structure as discussed herein.

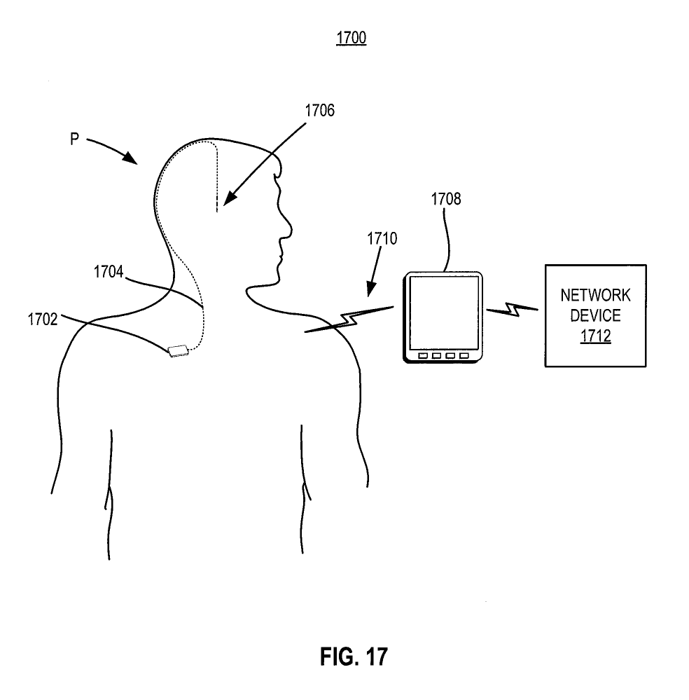

FIG. 17 illustrates an example of a medical system 1700 including a stimulation device 1702 and implantable stimulation lead 1704. For example, the stimulation device 1702 may include or be communicatively coupled to an implantable lead 1704 that is routed to a deep brain location of the patient P.

The stimulation device 1702 includes a signal generator and signal driver circuits (not shown) for generating stimulation signals. The output of the signal generator is coupled to the implantable lead 1704, such that stimulation energy is radiated at the designated stimulation sites in the patient P via electrodes 1706 of the implantable lead 1704.

The stimulation device 1702 may include a processing system (not shown) that controls the application of stimulation. In a typical implementation, stimulation is controlled based on signal attributes and timing parameters programmed into a memory device (not shown). Examples of the stimulation signal attributes include amplitude, frequency, pulse shape, and direction.

The stimulation parameters may be specified in various ways. For example, stimulation parameters may be downloaded into the stimulation device 1702 or may be predefined (e.g., programmed into the stimulation device 1702 during manufacture).

In the former case, the stimulation device 1702 communicates with an external device 1708 as shown in FIG. 17. The stimulation device 1702 and the external device 1708 may communicate with one another via a wireless communication link 1710 (as represented by the depicted wireless symbol).

The external device 1708 may communicate with another device (e.g., that may provide a more convenient means for a physician or other personnel to program the stimulation device 1702 or review information uploaded from the stimulation device 1702). For example, personnel may use a network device 1712 to program the stimulation device 1702 (e.g., to program stimulation parameters, to directly control stimulation, etc.).

It should be appreciated that various modifications may be incorporated into the disclosed embodiments based on the teachings herein. For example, the structure and functionality taught herein may be incorporated into types of devices other than the specific types of devices described above. Also, a lead may be constructed of materials and include components other than those specifically described herein.

In addition, a lead as taught herein may be used for applications other than DBS. For example, such a lead may be configured and implanted to stimulate various sections of the nervous system of a patient, or to stimulate other types of tissue (e.g., cardiac tissue, etc.).

Different embodiments of an apparatus (e.g., device) as taught herein may include a variety of hardware and software processing components. In some embodiments, hardware components such as processors, controllers, state machines, logic, or some combination of these components, may be used to implement one or more of the described components, circuits, or functions. For example, a hardware component may control the stimulation signal parameters (e.g., timing, duration, amplitude, pulse shape, electrode selection, etc.). In some implementations, such a hardware component comprises a processing system such as, for example, a processor device, a controller, an application specific integrated circuit (ASIC), or a system on a chip (SoC).

In some embodiments, code including instructions (e.g., software, firmware, middleware, etc.) may be executed on one or more processing devices to implement one or more of the described functions or components. The code and associated components (e.g., data structures and other components used by the code or used to execute the code) may be stored in an appropriate data memory that is readable by a processing device (e.g., commonly referred to as a computer-readable medium). For example, software executing on a processing system may control the stimulation signal parameters (e.g., timing, duration, amplitude, pulse shape, electrode selection, etc.).

Some of the operations described herein may be performed by a device that is located externally with respect to the body of the patient. For example, an external device may generate stimulation signals that are then coupled to an implanted lead.

The components and functions described herein may be connected or coupled in many different ways. The manner in which this is done may depend, in part, on whether and how the components are separated from the other components. In some embodiments, some of the connections or couplings represented by the lead lines in the drawings may be in an integrated circuit, on a circuit board or implemented as discrete wires or in other ways.

The signals discussed herein may take various forms. For example, in some embodiments a signal may comprise electrical signals transmitted over a wire, RF waves transmitted through a medium such as air, light pulses transmitted through an optical medium such as an optical fiber or air, and so on. In addition, a plurality of signals may be collectively referred to as a signal herein. The signals discussed above also may take the form of data. For example, in some embodiments an application program may send a signal to another application program. Such a signal may be stored in a data memory.

The recited order of the blocks in the processes disclosed herein is simply an example of a suitable approach. Thus, operations associated with such blocks may be rearranged while remaining within the scope of the present disclosure. Similarly, the accompanying method claims present operations in a sample order, and are not necessarily limited to the specific order presented.

Also, it should be understood that any reference to elements herein using a designation such as “first,” “second,” and so forth does not generally limit the quantity or order of those elements. Rather, these designations may be used herein as a convenient method of distinguishing between two or more different elements or instances of an element. Thus, a reference to first and second elements does not mean that only two elements may be employed there or that the first element must precede the second element in some manner. Also, unless stated otherwise, a set of elements may comprise one or more elements. In addition, terminology of the form “at least one of A, B, or C” or “one or more of A, B, or C” or “at least one of the group consisting of A, B, and C” used in the description or the claims means “A or B or C or any combination of these elements.” For example, this terminology may include A, or B, or C, or A and B, or A and C, or A and B and C, or 2A, or 2B, or 2C, and so on.

As used herein, the term “determining” encompasses a wide variety of actions. For example, “determining” may include calculating, computing, processing, deriving, investigating, looking up (e.g., looking up in a table, a database or another data structure), ascertaining, and the like. Also, “determining” may include receiving (e.g., receiving information), accessing (e.g., accessing data in a memory), and the like. Also, “determining” may include resolving, selecting, choosing, establishing, and the like.

It will further be understood that terms such as “top,” “bottom,” “above,” and “below” as used within the specification and the claims herein are terms of convenience that denote the spatial relationships of parts relative to each other rather than to any specific spatial or gravitational orientation. Thus, the terms are intended to encompass an assembly of component parts regardless of whether the assembly is oriented in the particular orientation shown in the drawings and described in the specification, upside down from that orientation, or any other rotational variation.

In some aspects, an apparatus or any component of an apparatus may be configured to (or operable to or adapted to) provide functionality as taught herein. This may be achieved, for example: by manufacturing (e.g., fabricating) the apparatus or component so that it will provide the functionality; by programming the apparatus or component so that it will provide the functionality; or through the use of some other suitable implementation technique.

While certain embodiments have been described above in detail and shown in the accompanying drawings, it is to be understood that such embodiments are merely illustrative of and not restrictive of the teachings herein. In particular, it should be recognized that the teachings herein apply to a wide variety of apparatuses and methods. It will thus be recognized that various modifications may be made to the illustrated embodiments or other embodiments, without departing from the broad scope thereof. In view of the above, it will be understood that the teachings herein are intended to cover any changes, adaptations or modifications which are within the scope of the disclosure.

What is claimed is:

1. An electrode structure, comprising:
a hollow cylindrical body comprising an electrically conductive metal, wherein:
the body defines a plurality of holes, such that a middle section and end sections of the body are delimited, at least in part, by the holes;
each of the end sections has an interior diameter that is greater than an interior diameter of the middle section;
the middle section comprises a plurality of electrode elements separated by the holes; and
each electrode element comprises a top surface and two side edges.

2. The electrode structure of claim 1, wherein each of the side edges of each of the electrode elements comprises a recessed ledge that has a top surface that is below an adjacent portion of the top surface of the electrode element.

3. The electrode structure of claim 2, wherein, for each of the recessed ledges of each of the electrode elements, the top surface of the recessed ledge forms a step down with respect to an adjacent portion of the top surface of the electrode element.

4. The electrode structure of claim 2, wherein, for each of the recessed ledges of each of the electrode elements, the recessed ledge slopes down from an adjacent portion of the top surface of the electrode element.

5. The electrode structure of claim 1, wherein each of the holes comprises a slot that runs in a longitudinal direction along the body.

6. The electrode structure of claim 1, wherein the body comprises a unitary body.

7. The electrode structure of claim 1, wherein the body comprises a biocompatible metal.

8. The electrode structure of claim 1, wherein the body has an outer circumference of 1.5 millimeters or less.

9. An implantable lead, comprising:
a lead body comprising insulation material; and
a plurality of electrode elements embedded at least partially within the insulation material, wherein:
each electrode element comprises a top surface, two side edges, and two arcuate end edges,
each side edge of each electrode element comprises a recessed ledge that has a top surface that is below an adjacent portion of the top surface of the electrode element, and
for each of the recessed ledges, at least a portion of the insulation material lies between the top surface of the recessed ledge and an outer surface of the lead body.

10. The implantable lead of claim 9, wherein:
the implantable lead further comprises a cylindrical spindle; and
the electrode elements lie on an outer surface of the spindle.

11. The implantable lead of claim 10, wherein:
the implantable lead further comprises a plurality of electrical conductors connected to the electrode elements;
the spindle defines at least one channel running longitudinally along an outer portion of the spindle; and
the electrical conductors are routed through the at least one channel.

12. The implantable lead of claim 9, wherein the lead body has an outer circumference of 1.5 millimeters or less.

13. The implantable lead of claim 9, wherein, for each of the recessed ledges of each of the electrode elements, the top surface of the recessed ledge forms a step down with respect to an adjacent portion of the top surface of the electrode element.

14. The implantable lead of claim 9, wherein, for each of the recessed ledges of each of the electrode elements, the recessed ledge slopes down from an adjacent portion of the top surface of the electrode element.

15. A method of manufacturing an implantable lead based on a cylindrical electrode structure that comprises a middle section and end sections, the method comprising:
removing a portion of an interior surface of each of the end sections, such that each of the end sections has an interior diameter that is greater than an interior diameter of the middle section;
forming a plurality of holes in the middle section, such that the middle section comprises a plurality of electrode elements separated by the holes, wherein each electrode element comprises a top surface and two side edges;
for each of the electrode elements, connecting an electrical conductor to the electrode element;
forming a lead body by embedding the electrode structure, at least in part, in an insulation material; and
centerless grinding the embedded electrode structure, such that the end sections are removed from the embedded electrode structure.

16. The method of claim 15, further comprising:
for each of the side edges of each of the electrode elements, removing a portion of the side edge to form a recessed ledge having a top surface that is below an adjacent portion of the top surface of the electrode element;
wherein the formation of the lead body comprises embedding the electrode structure, at least in part, in the insulation material such that, for each of the recessed ledges, at least a portion of the insulation material lies between the top surface of the recessed ledge and an outer surface of the lead body.

17. The method of claim 16, wherein:
the removal of the portion of an interior surface comprises milling the portion of the interior surface; and
the removal of the portion of the side edge comprises milling the portion of the side edge.

18. The method of claim 15, further comprising sliding the electrode structure onto a cylindrical spindle, wherein the embedding of the electrode structure is performed when the electrode structure is on the spindle.

19. The method of claim 18, wherein the embedding of the electrode structure comprises reflowing the insulation material over the electrode structure.

20. The method of claim 18, further comprising routing the electrical conductors via at least one channel formed in an outer portion of the spindle.

21. The method of claim 15, wherein the embedding of the electrode structure comprises:
placing the electrode structure into a mold; and
injecting the insulation material into the mold.

22. The method of claim 15, wherein the formation of the plurality of holes comprises milling a plurality of slots that run in a longitudinal direction along the cylindrical electrode structure.
